(19) United States
(12) Patent Application Publication
(10) Pub. No.: US 2013/0190702 A1
(43) Pub. Date: Jul. 25, 2013

(54) MEDICAL APPLICATOR DEVICE

(71) Applicants: Paul Longo, Portland, OR (US); Andrew Barofsky, Lake Oswego, OR (US); Wayne Phillips, Hudson, WI (US)

(72) Inventors: Paul Longo, Portland, OR (US); Andrew Barofsky, Lake Oswego, OR (US); Wayne Phillips, Hudson, WI (US)

(21) Appl. No.: 13/584,724

(22) Filed: Aug. 13, 2012

Related U.S. Application Data
(60) Provisional application No. 61/523,024, filed on Aug. 12, 2011.

(51) Int. Cl. A61M 35/00

(52) U.S. Cl. CPC A61M 35/003
USPC 604/290; 604/310; 604/311

(57) ABSTRACT

Embodiments provide a medical device in the form of an applicator for applying a medical composition to a patient. The applicator includes a body, a tip, and a delivery mechanism. The body defines a storage chamber for the medical composition and has an opening for deploying the medical composition from the storage chamber. The tip covers the opening and has a closed state wherein the tip maintains the medical composition in the storage chamber and an open state wherein the medical composition can be deployed through the opening and the tip.

### CROSS REFERENCE TO RELATED APPLICATIONS

The present application claims priority to U.S. Provisional Patent Application No. 61/523,024, filed Aug. 12, 2012, entitled “Medical Applicator Device,” the entire disclosure of which is hereby incorporated by reference in its entirety.

### GOVERNMENT INTERESTS

This invention was made with United States government support pursuant to various contracts from the United States Special Operations Command. The government has certain rights in the invention.

### TECHNICAL FIELD

Embodiments herein relate to medical devices for applying a medical composition to a patient and, in more particular applications, to such medical devices and methods for controlling bleeding and treating wounds.

### BACKGROUND

A leading cause of preventable battlefield death is non-compressible, intracavitary bleeding. Projectiles from weapons and improvised explosive devices frequently create small entrance wounds having limited or no visibility to the sites of non-compressible, intracavitary bleeding. Although several wound dressing technologies are being marketed to control aggressive hemorrhages from severe external injuries, these devices are particularly ineffective against narrow-entry wounds and the survival of the soldier is entirely dependent on immediate access to blood products and emergent surgical repair.

A principal method for treating bleeding wounds is to stop the flow of blood by applying pressure with a bandage to facilitate formation of a clot. Current wound dressings are often too stiff and too rigid to fit into a narrow space of a cavity wound or, if sufficiently pliable, do not adequately conform to irregular tissue geometries to cause rapid and effective hemostasis.

Granular and powder based hemostatic products have been employed to address the deficiency of current wound dressings for non-compressible wounds; however, these products also have significant drawbacks. Hemostats in the form of powders, particulates or granules pose an unacceptable risk in forming emboli, are difficult to deploy in austere environments (e.g., environments that include wind, darkness, etc.), are susceptible to washing or migration away from the wound site, and are difficult to retrieve from the wound site at a place of definitive care. Additionally, granular and powder based hemostatic products are difficult to handle because they may have high electrostatic charge causing them to stick to instruments, gloves and tissues, thus preventing adequate penetration into irregular wound cavities. Also, in windy environments, powders or granules may be very difficult to get into the wound and may actually blow back into a caregiver's eyes. Powder or granule based hemostats also exhibit a lack of physical cohesion, making them unable to sufficiently withstand the chaotic fluid environments created by severe, high pressure bleeding. Thus, these granular and powder based hemostats may simply wash away before effectively contributing to hemostasis.

One effective way to treat noncompressible hemorrhagic injuries is disclosed in U.S. patent application Ser. No. 12/773,680, filed May 4, 2010 and entitled “Hemorrhage Control Devices and Methods”, the disclosure of which is incorporated herein by reference in its entirety. The Ser. No. 12/773,680 application discloses hemostatic compositions in the form of a plurality of liquid expandable articles that have proven to be very successful in treating non-compressible hemorrhagic injuries. The Ser. No. 12/773,680 application also discloses a suitable medical applicator device.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 is a perspective view of a medical applicator device in accordance with embodiments herein;

FIG. 2 is another perspective view of the medical applicator device of FIG. 1 in accordance with various embodiments;

FIG. 3 is a side elevational view of the medical applicator device of FIG. 1 showing the device in a compact position or state in accordance with various embodiments;

FIG. 4 is a side elevational view of the device of FIG. 1, but showing the device in a cocked position or state in accordance with various embodiments;

FIG. 5 is an exploded perspective view of the device of FIG. 1 in accordance with various embodiments;

FIG. 6 is a cross-sectional view of the device of FIG. 1 in accordance with various embodiments;

FIG. 7 is another cross-sectional view of the device of FIG. 1 in accordance with various embodiments;

FIG. 8 is another cross-sectional view of the device of FIG. 1 showing the device in a deployed position or state, with the exception of a tip component that is shown in a closed position rather than an open condition that would exist in the deployed position, in accordance with various embodiments;

FIG. 9 is a front view of a tip component of a medical applicator device in accordance with various embodiments;

FIG. 10 is a side elevational view taken from line 10-10 in FIG. 9 in accordance with various embodiments;

FIG. 11 is a back view of the tip component of FIG. 9 in accordance with various embodiments;

FIG. 12A is an enlarged cross-sectional view taken along line 12-12 in FIG. 9 in accordance with various embodiments;

FIG. 12B is an enlarged perspective view of the tip component of FIG. 9 in accordance with various embodiments;

FIG. 13 is a perspective view of the tip component of FIG. 9 in accordance with various embodiments;

FIG. 14 is another perspective view of the tip component of FIG. 9 in accordance with various embodiments;

FIG. 15 is a cross-sectional view of a body component of a medical applicator device in accordance with various embodiments;

FIG. 16 is front view of the body component of FIG. 15 in accordance with various embodiments;

FIG. 17 is a back view of the body component of FIG. 15 in accordance with various embodiments;

FIG. 18 is a cross-sectional view taken along line 18-18 in FIG. 15 in accordance with various embodiments;

FIG. 19 is an enlarged view of the portion of the body component encircled by line 19-19 in FIG. 15 in accordance with various embodiments;

FIG. 20 is a perspective view of the body component of FIG. 15 in accordance with various embodiments;

FIG. 21 is another perspective view of the body component of FIG. 15 in accordance with various embodiments;

FIG. 22 is a side elevational view of a collet component of a medical applicator device in accordance with various embodiments;

FIG. 23 is a back view of the collet of FIG. 22 in accordance with various embodiments;

FIG. 24 is a front view of the collet of FIG. 22 in accordance with various embodiments;

FIG. 25 is a cross-sectional view taken along the line 25-25 in FIG. 23 in accordance with various embodiments;

FIG. 26 is a perspective view of the collet of FIG. 22 in accordance with various embodiments;

FIG. 27 is another perspective view of the collet of FIG. 22 in accordance with various embodiments;

FIG. 28 is a side elevational view of a handle component of a medical applicator device in accordance with various embodiments;

FIG. 29 is a back view of the handle of FIG. 28 in accordance with various embodiments;

FIG. 30 is a front view of the handle of FIG. 28 in accordance with various embodiments;

FIG. 31 is a cross-sectional view taken along line 31-31 in FIG. 29 in accordance with various embodiments;

FIG. 32 is a perspective view of the handle of FIG. 28 in accordance with various embodiments;

FIG. 33 is another perspective view of the handle of FIG. 28 in accordance with various embodiments;

FIG. 34 is front view of a piston of a medical applicator device in accordance with various embodiments;

FIG. 35A is a back view of the piston of FIG. 34 in accordance with various embodiments;

FIG. 35B is an alternative back view of a piston in accordance with various embodiments;

FIG. 36A is a cross-sectional view taken along line 36-36 in FIG. 35 in accordance with various embodiments;

FIG. 36B is an alternative cross-sectional view of a piston in accordance with various embodiments;

FIG. 37A is a cross-sectional view taken along line 37-37 in FIG. 36 in accordance with various embodiments;

FIG. 37B is an alternative cross-sectional view of a piston in accordance with various embodiments;

FIG. 38 is a perspective view of the piston of FIG. 34;

FIG. 39 is another perspective view of the piston of FIG. 34 in accordance with various embodiments; and

FIG. 40 is a front view of a tip component in an open condition or state in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Embodiments herein provide hemostatic compositions and devices, as well as methods of manufacturing such compositions and applicator devices. In addition, various embodiments include methods for treating hemorrhagic injuries.

In an embodiment, a medical device is provided in the form of an applicator for applying a medical composition to a patient. The applicator includes a body, a tip, and a delivery mechanism. The body defines a storage chamber for the medical composition and has an opening for deploying the medical composition from the storage chamber. The tip covers the opening and has a closed state wherein the tip maintains the medical composition in the storage chamber and an open state wherein the medical composition can be deployed through the opening and the tip.

In an embodiment, the delivery mechanism is a telescoping delivery mechanism that is moveable between a compact position for storage and transport and a cocked position for deployment of the medical composition from the applicator.

The telescoping delivery mechanism may include a piston and a user interface in the form of an ergonomic handle. The piston may be located in the storage chamber to deploy the medical composition stored in the chamber through the opening and the tip as the piston moves toward the opening from a stored position spaced from the opening. In an embodiment, the handle has a compact position and a cocked position. In the compact position, the handle may be located adjacent the body with the piston located in the stored position. In the cocked position, the handle may be spaced from the body and in operable engagement with the piston in the stored position to move the piston from the stored position toward the opening in response to an actuating force applied to the handle by a user.

In an embodiment, the telescoping delivery mechanism further includes a rod connected to the handle for translating movement therewith, the rod having a first state wherein the rod is free to translate relative to the piston and a second state wherein the rod is connected to the piston to transmit an actuating force thereto from a user engaging the handle.

In an embodiment, the tip may have a convex shaped portion extending outwardly from the body and the opening. The convex shape of the tip may be a generally semi-spherical or bullet shape, and in another embodiment, may be a generally semi-spherical profile defined by a plurality of radially extending stiffening ribs. One or more of the stiffening ribs may have a frangible section in the form of a thinned section extending along the corresponding rib. In a further embodiment, the tip may include six of the stiffening ribs, with three of the stiffening ribs having the frangible section and three of the stiffening ribs being free of a frangible section. Alternatively, the tip may be formed of a plurality of deformable elements responsive to force applied by a user dispensing a medical composition. The term “deformable” is used broadly herein to include elastic or plastic deformation.

In an embodiment, the medical composition is a hemostatic composition comprising a plurality of liquid expandable articles capable of expanding upon contact with a liquid. In the following description, unless further particularized or otherwise noted, the term “liquid expandable” is intended to refer to any material or substance that expands upon contact with a liquid.

Thus, in an embodiment, there is an applicator device, comprising a body having a storage chamber for holding a composition, the storage chamber having an opening; and a delivery mechanism for dispensing the composition from the storage chamber through the opening in response to actuation by a user, the delivery mechanism comprising a delivery mechanism that is moveable between a compact position for storage and transport and a cocked position for deployment of the composition from the applicator.

In another embodiment, there is provided an applicator device, comprising a body having a storage chamber for holding a composition, the storage chamber having an opening; a tip covering the opening of the storage chamber, wherein the tip has a closed state in which the composition is maintained within the storage chamber and an open state in which the composition can be dispensed through the opening and the tip; and a delivery mechanism for dispensing the composition from the storage chamber through the opening in response to actuation by a user.

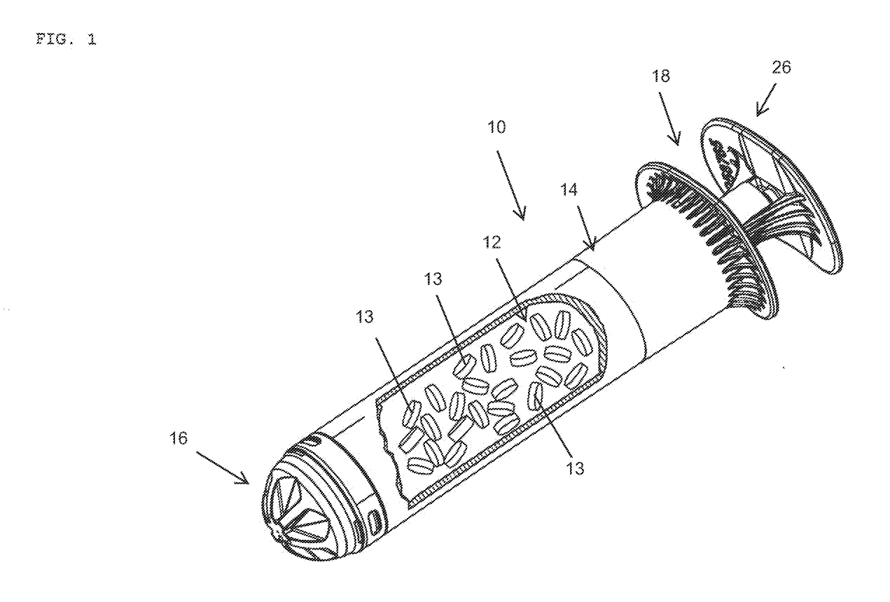

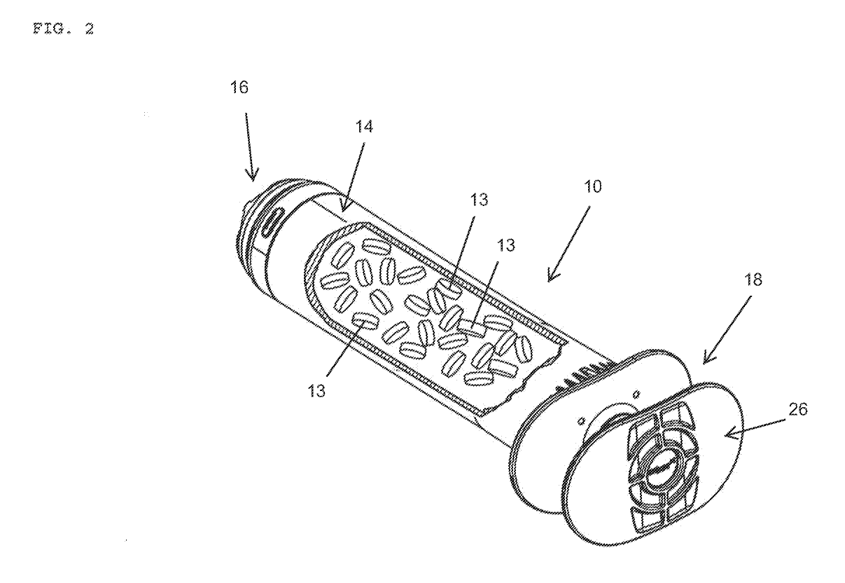

With reference to FIGS. 1-8, a medical device is shown in the form of an applicator 10 for applying a medical composition 12 to a patient and, in more specific cases, for applying a hemostatic composition to a bleeding wound of a patient. In this regard, a hemostatic composition may comprise a plurality of liquid expandable articles 13 capable of expanding upon contact with a liquid. Such a hemostatic composition is more fully described in Ser. No. 12/773,680, filed May 4, 2010, entitled “Hemorrhage Control Devices and Methods”, as well as discussed in the Background section of the present application. While an example medical composition is identified herein, it should be understood that the applicator 10 may be utilized with other medical compositions 12, as dictated by the needs of each particular application. Accordingly, no limitation to a particular medical composition should be inferred unless expressly recited in an appended claim.

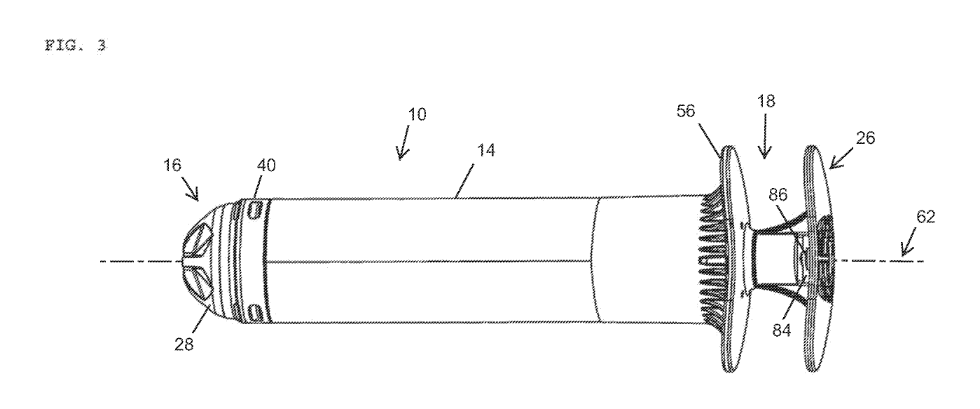

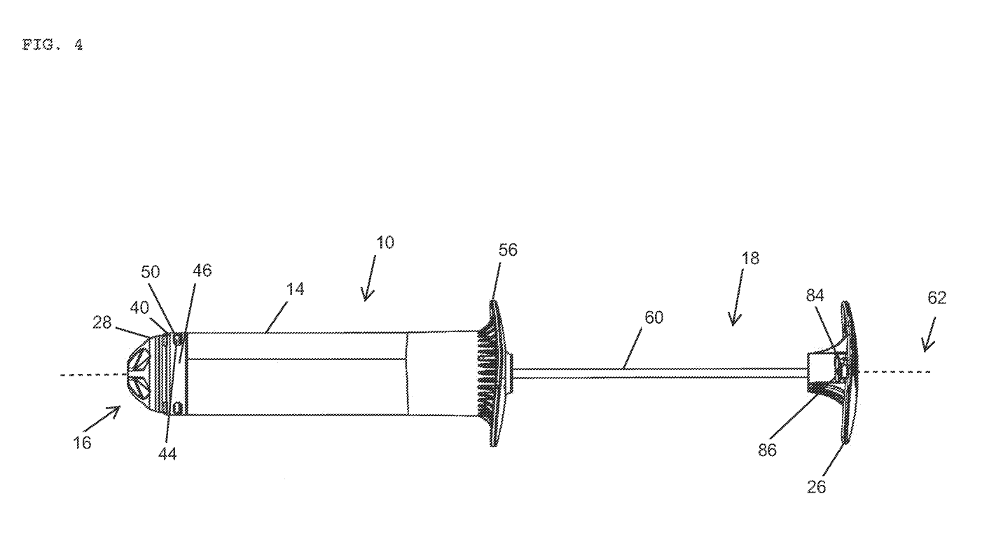

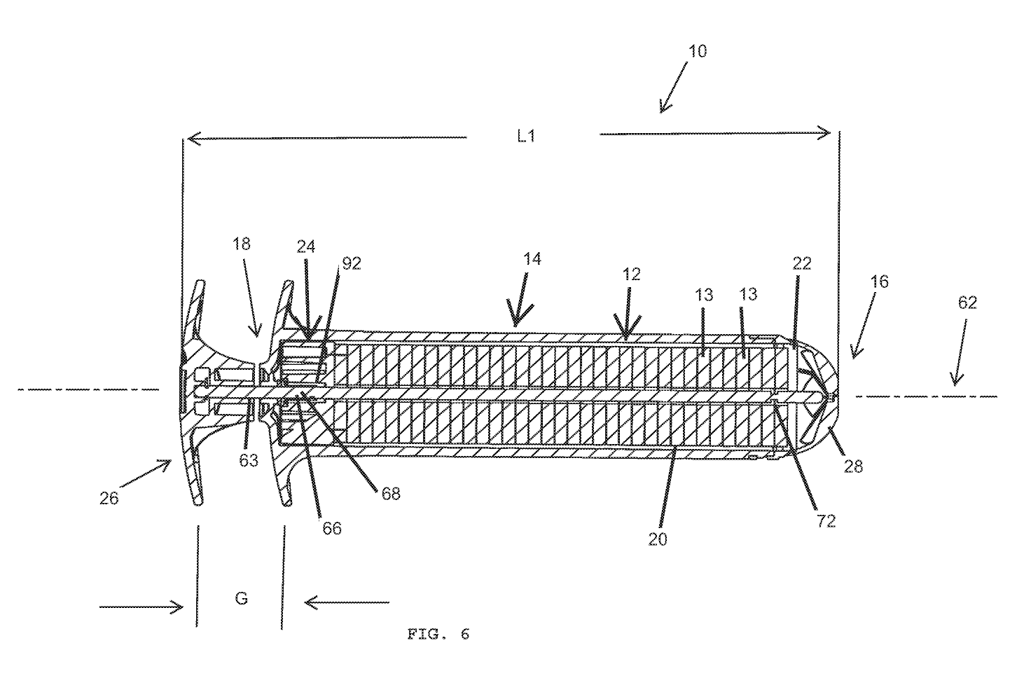

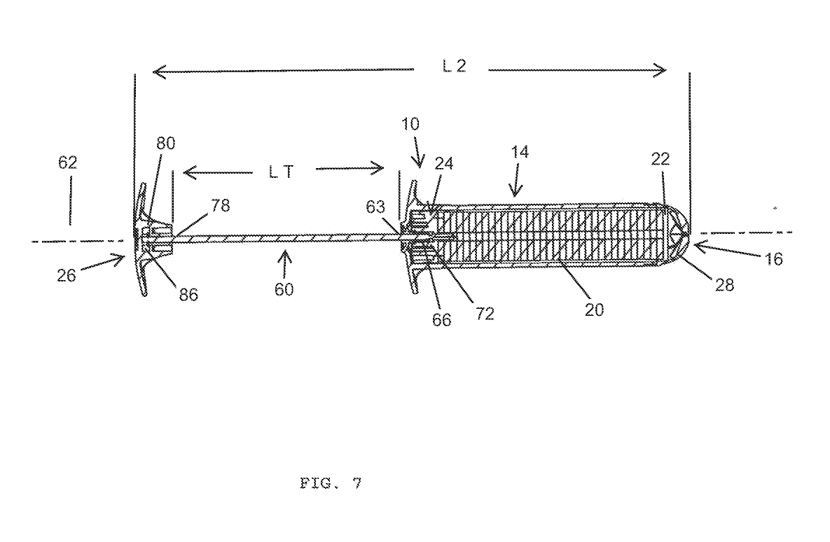

The applicator 10 includes a body 14, a tip 16, and a telescoping delivery mechanism 18. The body 14 defines a storage chamber 20 for the medical composition 12 and has an opening 22 for deploying the medical composition from the storage chamber 20. The tip 16 covers the opening 20 and has a closed state wherein the tip 16 maintains the medical composition 12 in the storage chamber 20 and an open state wherein the medical composition 12 can be deployed through the opening 22 and the tip 16. The telescoping delivery mechanism 18 includes a piston 24 and a user interface shown in the form of a handle 26. As best seen in FIG. 6, piston 24 is located in the storage chamber 20 to deploy the medical composition 12 stored in the chamber 20 through the opening 22 and the tip 16 as the piston 24 moves toward the opening from a stored position (shown in FIGS. 6 and 7) spaced from the opening 22 and the tip 16. Handle 26 has a compact position shown in FIGS. 3 and 6 and a cocked position shown in FIGS. 4 and 7. In the compact position, as shown in FIGS. 3 and 6, the handle 26 is located adjacent the body 14 with the piston 24 located in the stored position. In the cocked position, as shown in FIGS. 4 and 7, the handle 26 is spaced from the body 14 and operably engaged with the piston 24 in the stored position to move the piston 24 from the stored position toward the opening 22 and the tip 16 in response to an actuating force applied to the handle 26 by a user. For full deployment of all of the medical composition 12 from the storage chamber 20, the piston 24 and handle 26 are moved to the deployed position shown in FIG. 8.

With reference to FIGS. 6 and 7, the applicator 10 has a length L₁ in the compact position and a length L₂ in the cocked position that is greater than the length L₁ by a travel length L_{T} which represents the longitudinal distance traveled by the handle 26 as it moves from the compact position shown in FIG. 6 to the cocked position shown in FIG. 7. As shown in FIG. 8, the applicator has a length L₃ in the deployed position that is less than the length L₂ by a stroke length L_{S} which represents the longitudinal distance traveled by the piston 24 as it moves from the stored position shown in FIG. 7 to the deployed position in FIG. 8. Preferably, the applicator is constructed and sized so that L₃=L₁ and L_{T}=L_{S}.

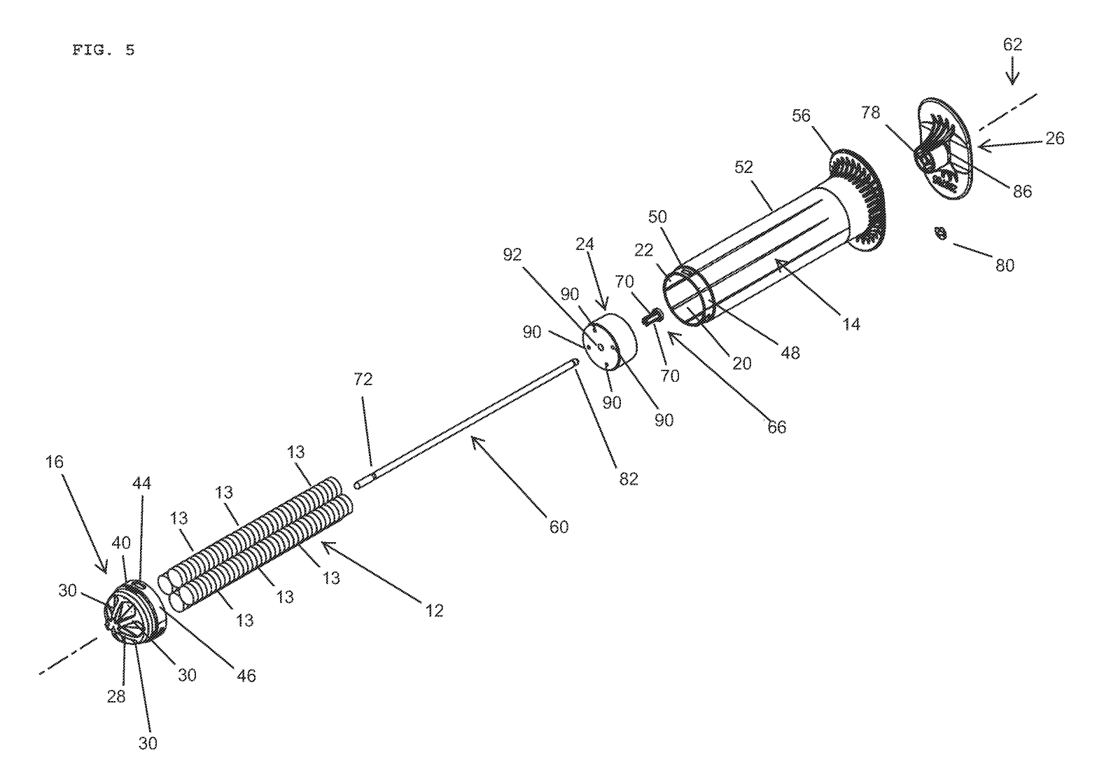

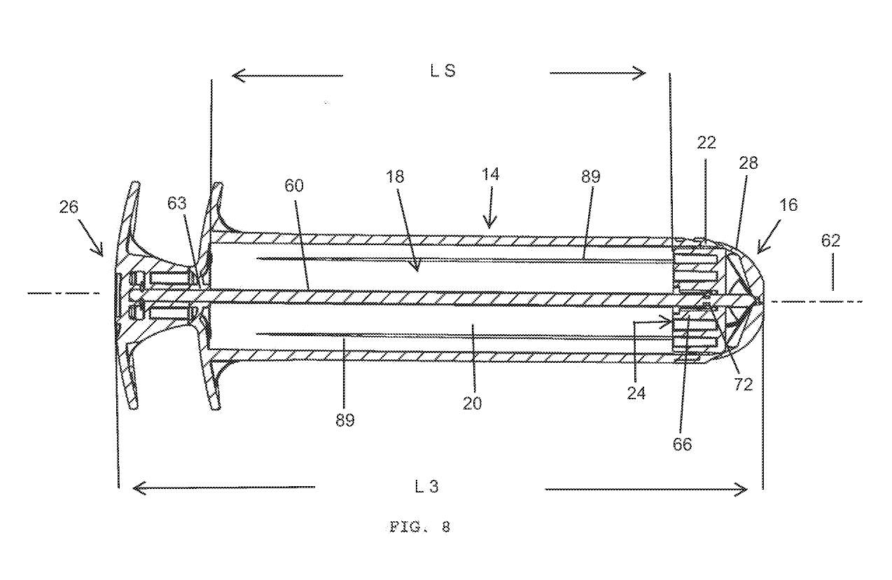

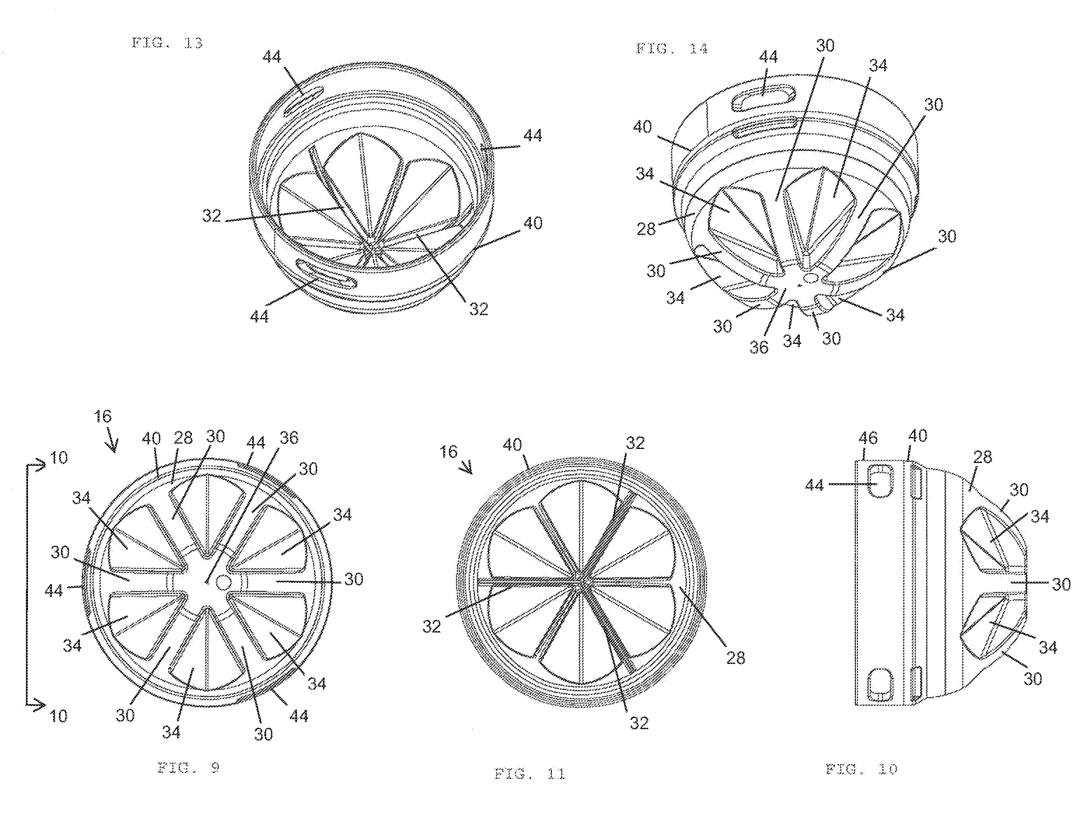

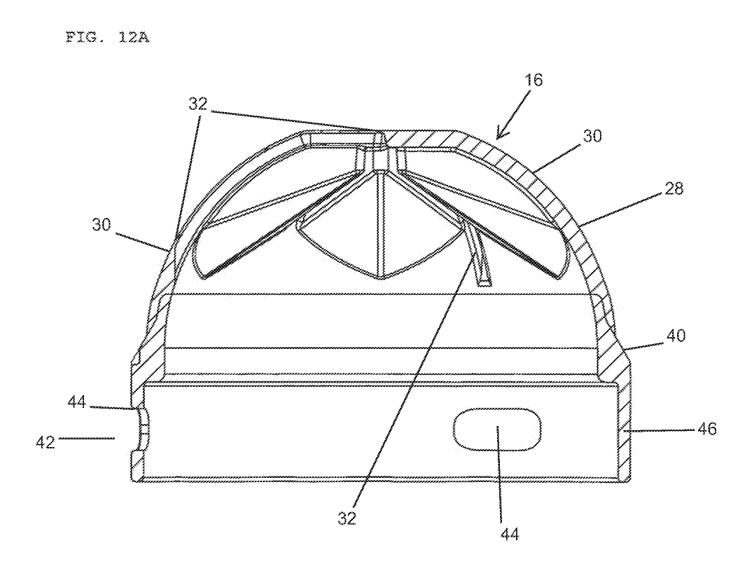

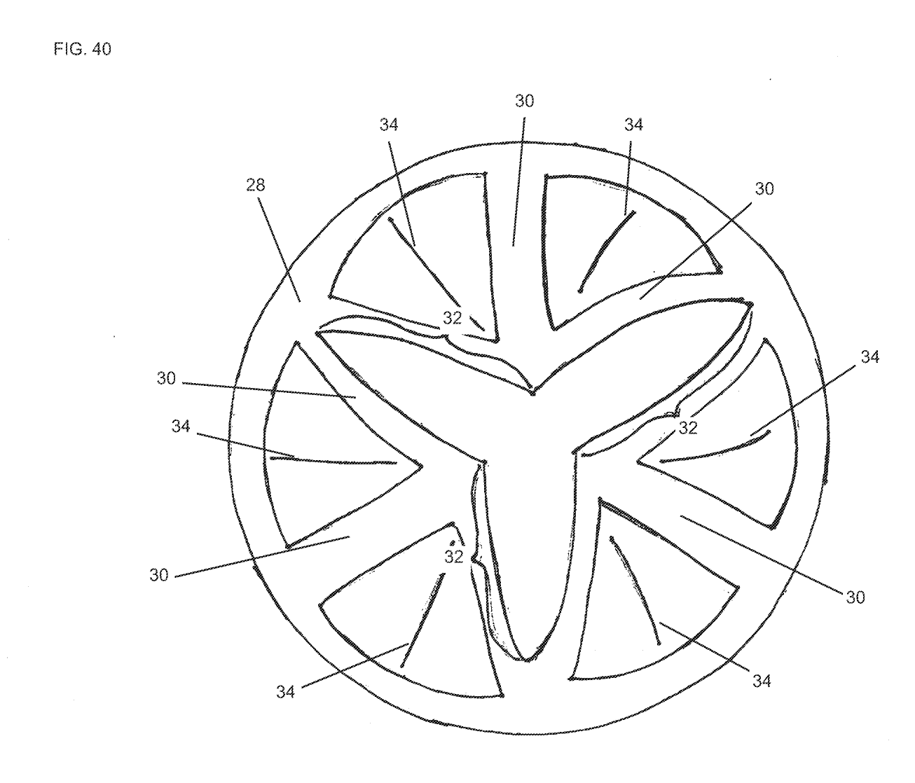

The tip 16 preferably has a deformable, convex shaped portion 28 extending outwardly from the body 14 and the opening 22. This convex shaped portion 28 assists in allowing the applicator 10 to be successfully located relative to a hemorrhagic injury, such as at least partially inserted into a hemorrhagic injury/wound, particularly one having a small entrance wound leading to cavity wound having non-compressible, intracavitary bleeding. In this regard, the convex shape of the tip 16 is generally semi-spherical or bullet shaped, and even more preferably is a generally semi-spherical profile defined by a plurality of radially extending stiffening ribs 30, as best seen in FIGS. 5 and 9-14. In some embodiments, to insure that the convex portion 28 is deformable without requiring undue force, one or more of the stiffening ribs 30 has a thinned (frangible) section 32 extending along the corresponding rib 30, as best seen in FIG. 12A. FIG. 40 shows the tip 16 in an open condition or state after the sections 32 of the portion 28 have been torn/separated by deployment of the medical composition 12 from the applicator 10. In the illustrated embodiment, the tip 16 includes six of the stiffening ribs 30, with three of the stiffening ribs 30 having thinned section 32 and three of the stiffening ribs 30 being free of thinned section 32. The illustrated stiffening ribs 30 are angularly spaced from each other by pie-shaped reliefs 34 (as seen, for example, in FIG. 9), with the angular spacing between each of the ribs 30 being equal. It should be understood that in some embodiments, the tip 16 can include more than six ribs 30 or fewer than six ribs 30 and that all of ribs 30, none of ribs 30, or any number of ribs 30 may include a thinned section 32. It should also be understood that in some embodiments, the angular spacing between ribs 30 need not be equal. In one embodiment, a small opening 36 is provided at a central location on the tip 16 and acts as an initiation point for the separation/tearing of thinned sections 32. Portion 28 is preferably injection molded and may be formed of any suitable material, with one preferred material being a liquid silicone rubber.

Thinned sections 32 may be frangible and, as such, may be formed of a thinned wall of material that may be torn or separated by application of force by a user. Alternatively, the sections may be coupled by a seam formed by any suitable mechanism including adhesive, ultrasonic welding, etc. The seam between the sections may then be torn or separated by application of force by a user. In either situation, portion 28 is deformable/frangible and, upon application of force to portion 28, results in formation or propagation of an opening through which medical composition 12 may be dispensed.

In some embodiments such as shown in FIG. 12B, tip 16 includes radially thickened sections 38 to absorb crack propagation energy and reduce the likelihood of cracks extending beyond the thinned (deformable/frangible) sections 32. In this regard, radially thickened sections 38 include central channels (not shown) that align with the sections 32. In some embodiments, radially thickened sections 38 can be localized at the proximal ends of sections 32. In some embodiments, the radially thickened sections may extend circumferentially around the proximal base of tip 16.

As seen in FIGS. 12A and 12B, tip 16 includes a mount ring 40 formed of a rigid material, for example more rigid than portion 28. Mount ring 40 includes a connecting structure 42 for attaching tip 16 to body 14. This connecting structure 42 includes three equally, circumferentially spaced windows 44 located in a cylindrical portion 46 of the mount ring 40.

Cylindrical portion 46 is sized to fit over a closely conforming cylindrical portion 48 (see, for example, FIG. 5) of body 14 surrounding opening 22, with windows 44 engaging three equally circumferentially spaced projections 50 on portion 48 to connect mount ring 40 to body 14.

Mount ring 40 may be injection molded and may be formed of any suitable material, with one example material being polycarbonate. In an embodiment, portion 28 and mount ring 40 may be bonded to each other during a suitable molding process, such as an insert molding process, a bi-injection molding process, and/or a two shot molding process. It should be understood that many other suitable forms of connections may be provided between mount ring 40 and body 14, and that no limitation to the specific form shown herein is intended unless specifically stated in an appended claim. It should further be understood that in some embodiments it may be desirable for tip 16 to just include portion 28, with portion 28 being connected to body 14 using other suitable means, such as a suitable bonding agent or method.

FIGS. 9-14 and 40 show other views of tip 16, but it should be understood that the illustrated details may or may not be desired for any particular application and are highly dependent upon a number of parameters, including for example, the particular material(s) used for tip 16, the methods employed in forming tip 16, and the particular form and type of medical composition 12 to be deployed by applicator 10.

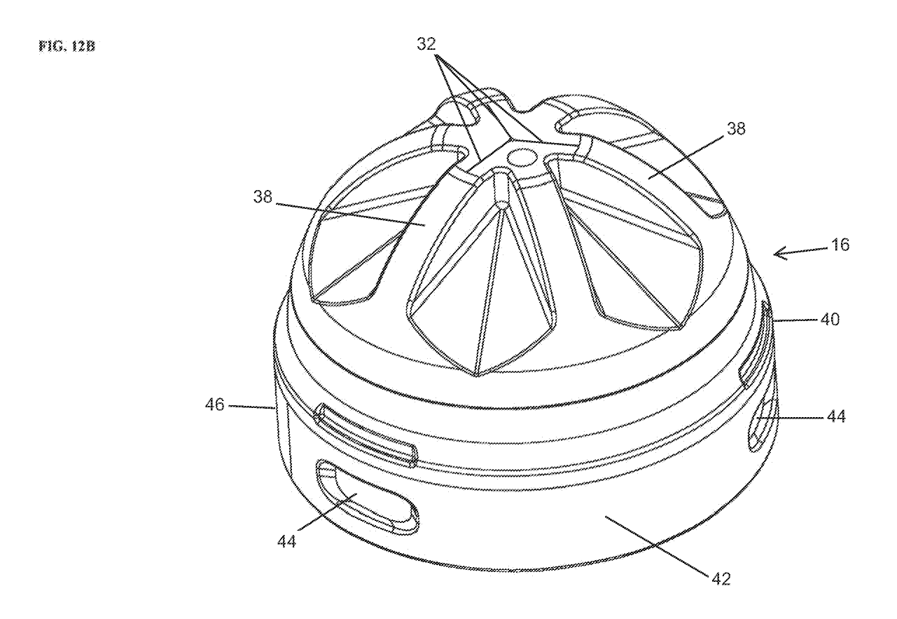

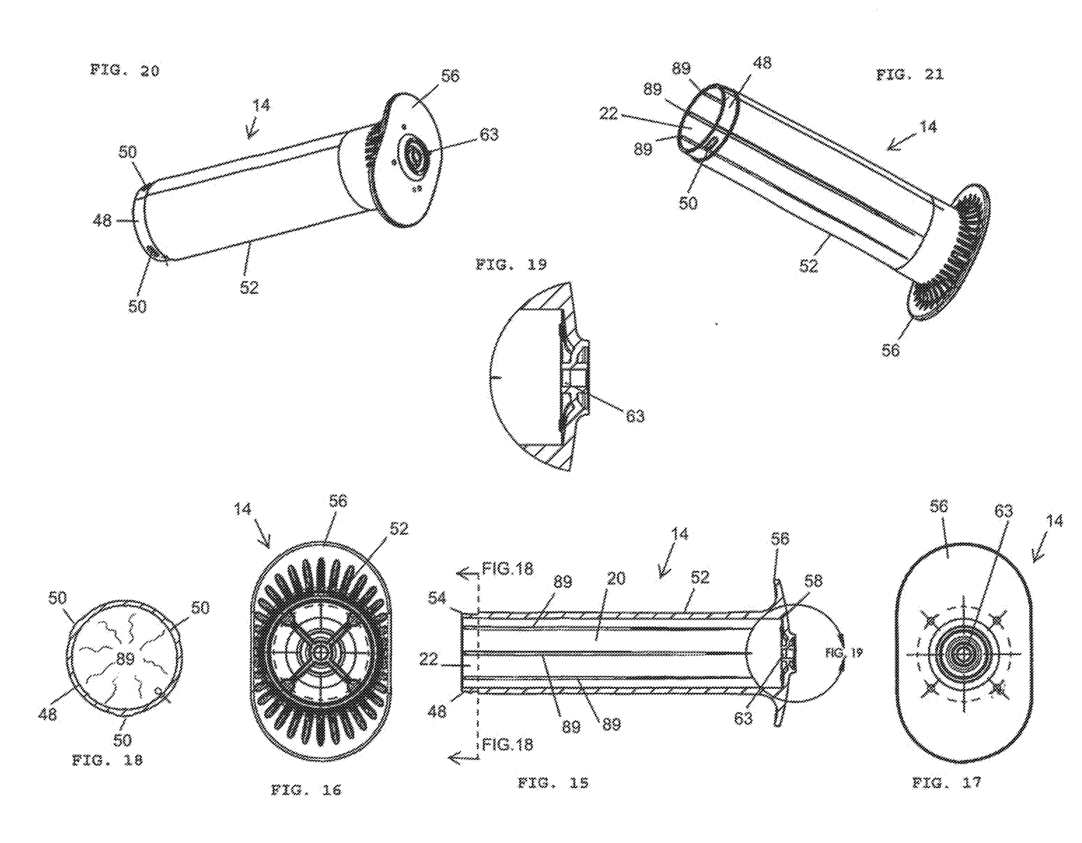

As seen for example in FIG. 15, body 14 is provided in the form of a cylindrical tube 52, with storage chamber 20 having a cylindrical shape defined by the interior of tube 52, opening 22 being located at one end 54 of tube 52, and a finger engageable shoulder structure 56 being located at an opposite end 58 of tube 52. Body 14 may be injection molded as a single piece using a suitable material, such as polycarbonate. In some embodiments, it may be desirable to include a light surface texture on the exterior of all or part of body 14 to reduce slippage, and a translucent color to indicate the fill level for medical composition 12 stored in storage compartment 20. While body 14 is shown as a single piece, it should be understood that multi-piece constructions may be desirable for some applications.

FIGS. 15-21 show other details of an embodiment of body 14, but it should be understood that these details may or may not be desired for any particular application and are highly dependent upon a number of parameters, including for example, the particular material(s) used for body 14, the methods employed in forming body 14, and the particular form and type of medical composition 12 to be deployed by applicator 10.

In an embodiment, a tip may fit on and/or around opening 22. Alternatively, a tip may fit at least partially within opening 22, such as in the form of a plug. An embodiment in the form of a plug may be formed of any suitable material, including a liquid expandable material. In an embodiment, a plug formed from a liquid expandable material may be the first of a plurality of liquid expandable articles, such that, when dispensed, the plug and the remaining liquid expandable articles are dispensed, such as into a wound.

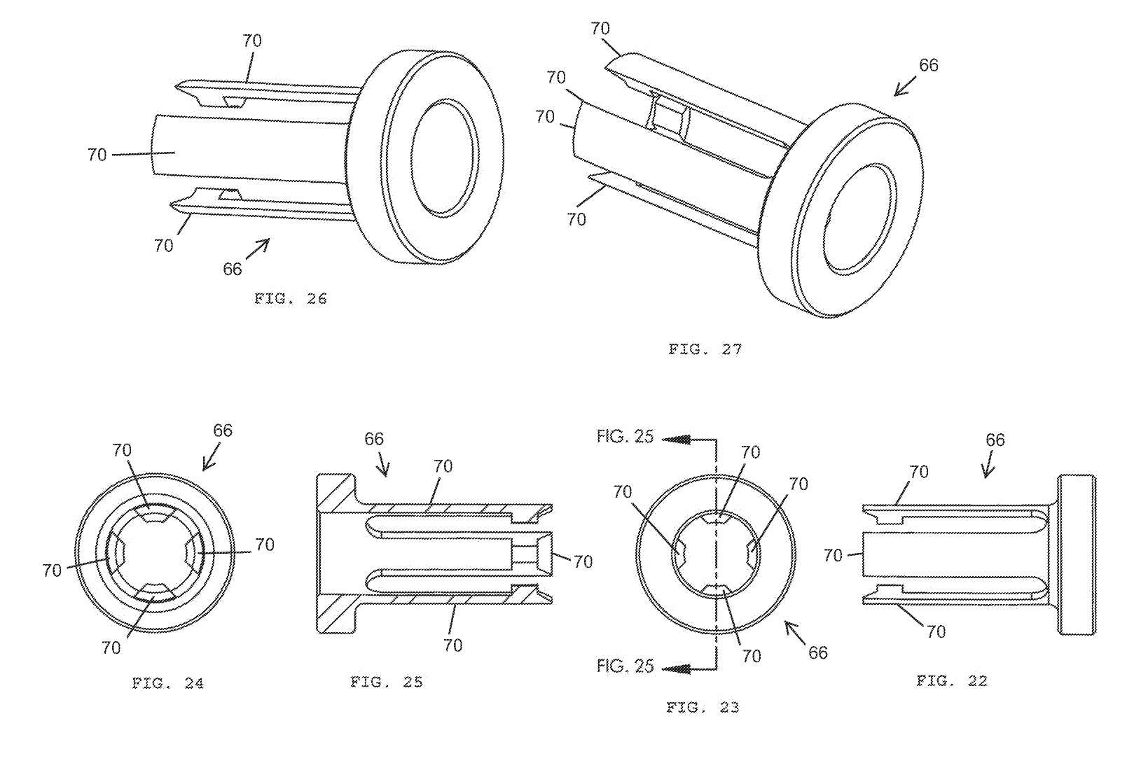

As seen for example in FIGS. 5-8, telescoping delivery mechanism 18 includes a cylindrical rod 60 connected to handle 26 for translating movement therewith along a central axis 62 between the compact position shown in FIG. 6 and the cocked position shown in FIG. 7. In this regard, rod 60 is guided for such translation by a closely conforming bore 63 provided in body 14, as seen for example in FIGS. 6-8 and 19. Rod 60 has a first state shown in FIG. 6 wherein handle 26 is in the compact position and rod 60 translates freely relative to piston 24, and a second state shown in FIG. 7 wherein handle 26 is in the cocked position and rod 60 is connected with piston 24 to transfer the actuating force from handle 26 to piston 24 for deployment of medical composition 12 from storage chamber 20 as piston 24, rod 60, and handle 26 translate together toward opening 22 and tip 16 along axis 62. In this regard, the telescoping delivery mechanism includes an engagement structure connecting piston 24 and rod 60 together for translating movement when handle 26 is moved from the compact position to the cocked position. The engagement structure includes a multi-fingered collet 66 that is fitted in a mating bore 68 of piston 24 so that fingers 70 (see for example FIGS. 22-27) of collet 66 engage an annular channel 72 (see FIG. 5, for example) formed on rod 60 to connect rod 60 and piston 24 for translating movement. In an embodiment, it is desirable to construct the engagement structure to provide audible and tactile feedback to the user when engaged. In an embodiment, collet 66 is positioned in an opened, spring-loaded state while in the stored position. When handle 26 is cocked, fingers 70 snap into annular channel 72 in the rod 60, producing an audible and tactile “click” that indicates the device is engaged and ready to deploy.

Rod 60 may be formed of any suitable material, such as machined type 304 stainless steel, and collet 66 may be formed of any suitable material, including any suitable injection-molded material. While collet 66 and annular channel 72 are shown, it should be understood that there are other suitable forms/constructions for the engagement structure that may be utilized in applicator 10, including for example, a cotter pin that engages a mating feature on rod 60, or an external retaining ring that engages a mating feature on rod 60. FIGS. 22-27 show other details of an embodiment of collet 66, but it should be understood that these details may or may not be desired for any particular application and are highly dependent upon a number of parameters, including for example, the particular material(s) used for the piston 24, rod 60, and collet 66, the methods employed in forming these components, and the particular form and type of medical composition 12 to be deployed by applicator 10.

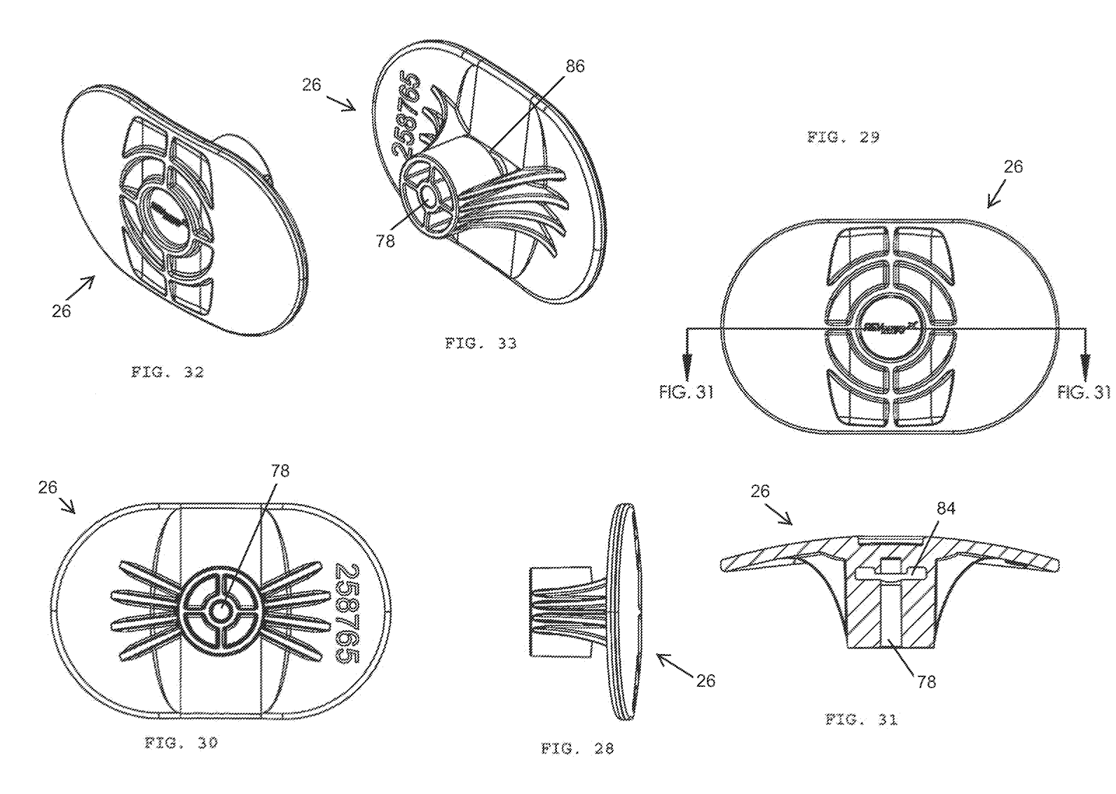

In an embodiment, handle 26 is attached to rod 60 so that handle 26 is rotatable relative to rod 60 about axis 62, while being fixed to rod 60 for translation along axis 62. In this regard, while any suitable attachment may be used, in the illustrated embodiment such as seen in FIGS. 5 and 7, rod 60 is received in a mating bore 78 in handle 26 and is attached to handle 26 with an external retaining ring 80, in the form of a C-clip, that is engaged in an annular channel 82 on rod 60 and retained in a receiving opening 84 of handle 26. As seen for example in FIGS. 3 and 33, retaining ring 80 can be inserted into opening 84 for engagement with channel 82 via a slot 86 formed in handle 26. As seen for example in FIG. 6, in the compact position, handle 26 is spaced from shoulder 56 on body 14 by a gap G sufficiently large to allow a user's fingers to be inserted between shoulder 56 and handle 26, including when the user is wearing a protective glove. Handle 26 may be injection molded as a one-piece component, and may be formed of any suitable material, such as polycarbonate. While handle 26 is shown as a one-piece component, it should be understood that multi-piece constructions of handle 26 may be desirable for some applications. FIGS. 28-33 show other details of an embodiment of handle 26, but it should be understood that these details may or may not be desired for any particular application and are highly dependent upon a number of parameters, including for example, the particular material(s) used for handle 26, the methods employed in forming handle 26, and the particular form and type of medical composition 12 to be deployed by applicator 10.

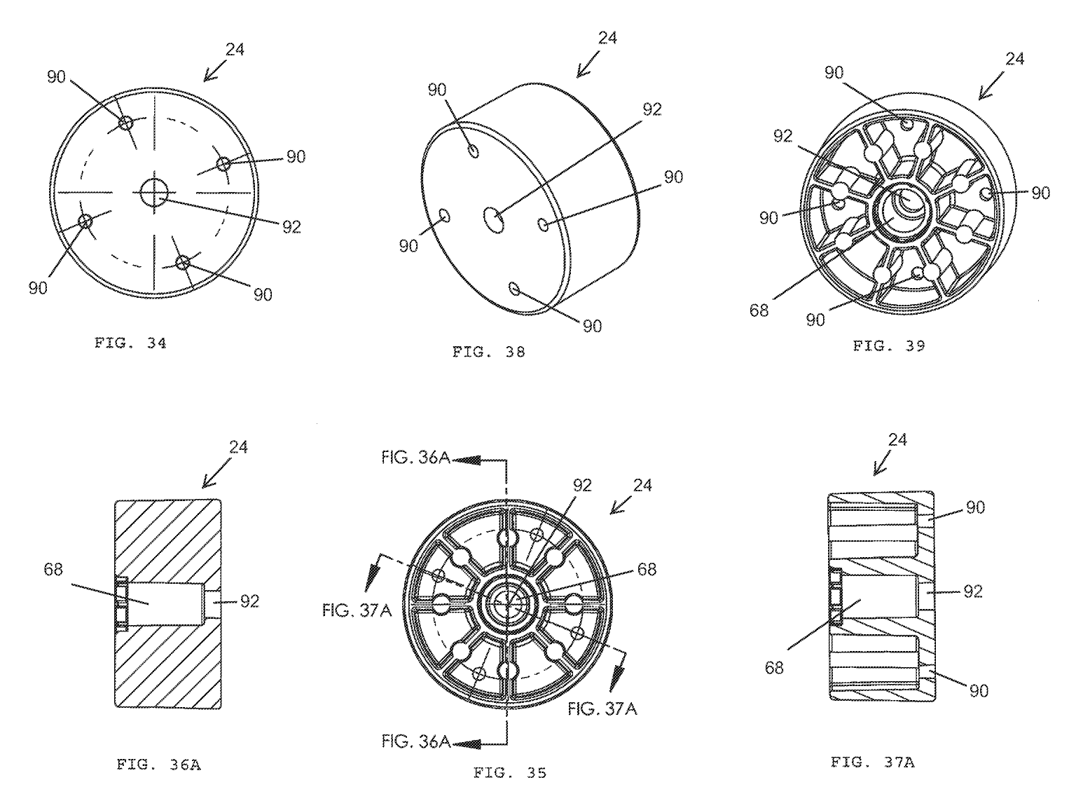

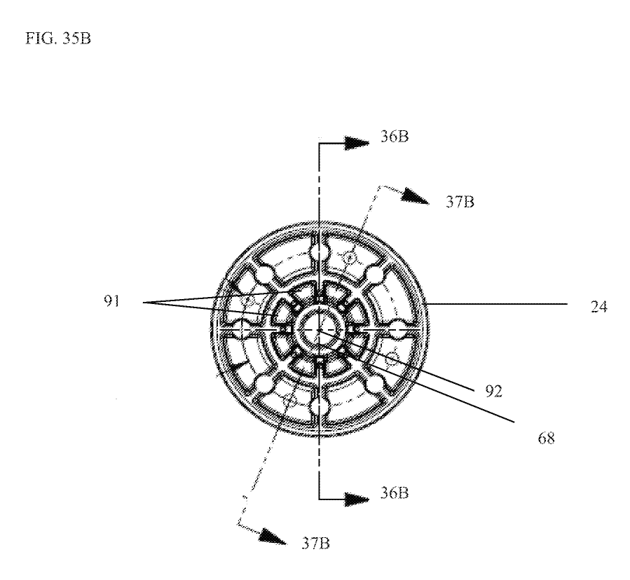

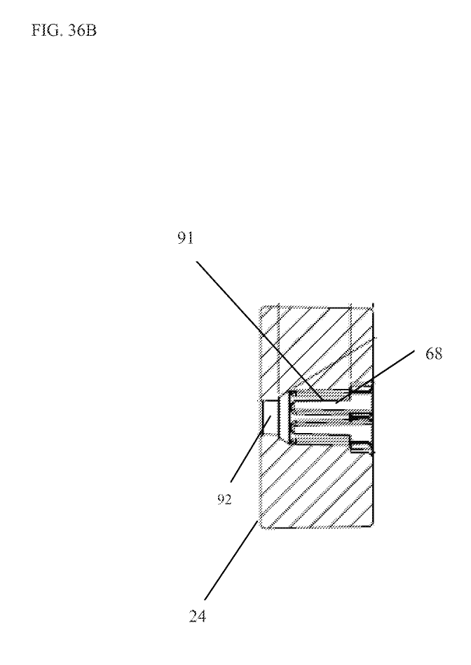

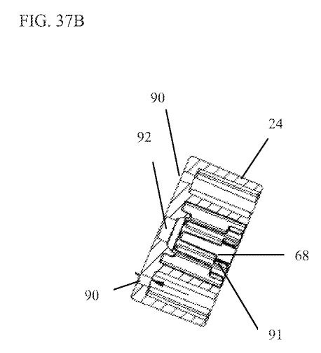

In accordance with various embodiments, piston 24 has a cylindrical shape that conforms to the interior of tube 52 to be guided thereby for translating movement along central axis 62 from the stored position shown in FIGS. 6 and 7 to the deployed position shown in FIG. 8. In this regard, the interior of tube 52 can be provided with a plurality of circumferentially spaced, longitudinally extending guide ribs 89 (see for example FIGS. 8, 15 and 21), with the piston 24 having a diameter selected to allow guided translation of the piston 24 by/along the ribs 89. As seen for example in FIGS. 5, 34, and 37, piston 24 is provided with a plurality of through holes 90 that prevent air pressure from building up on one side or the other of piston 24 as piston 24 translates in the interior chamber 18 along axis 62. Piston 24 includes a guide bore 92 that closely conforms to the exterior of rod 60 to guide rod 60 as it translates along axis 62 from the compact position shown in FIG. 6 to the cocked position shown in FIG. 7. Piston 24 may be injection molded as a one-piece component out of any suitable material, such as polycarbonate. While piston 24 is shown herein as a one-piece component, it may be desirable in some applications to form piston 24 as a multi-piece component. FIGS. 34-39 show other details of an embodiment of piston 24, but it should be understood that these details may or may not be desired for any particular application and are highly dependent upon a number of parameters, including for example, the particular material(s) used for piston 24, the methods employed in forming piston 24, and the particular form and type of medical composition 12 to be deployed by applicator 10.

As seen for example in FIGS. 35B, 36B, and 37B, guide ribs 91 may be resident in piston 24 and provide a tapered locking feature that secures collet fingers 70 during deployment to prevent them from springing radially outward and losing contact with annular engagement channel 72 in rod 60. During storage and cocking, this feature is positioned distal to collet fingers 70 to provide radial clearance. During deployment, the engaged collet 66 slides axially relative to piston 24 until collet fingers 70 are bounded by and secured in place by the tapered locking aspect of guide ribs 91.

In an embodiment, medical composition 12 may be loaded into storage compartment 20 via opening 22 prior to assembly of tip 16 onto body 14, but after piston 24, rod 60, and handle 26 are assembled with body 14 and placed in storage and compact positions, respectively. In this regard, it should be understood that while medical composition 12 is shown as four stacked columns, this is for illustration purposes only and the medical composition 12 may be loaded into the storage chamber 20 in an ordered or random arrangement. Once medical composition 12 is loaded into chamber 20, tip 16 can be assembled to body 14 to place applicator 10 in a usable condition. In operation, a user grasps handle 26 and moves handle 26 and rod 60 from the compact position to the cocked position. The user then inserts applicator 10 into a desired position in the wound, using tip 16 as required to aid in such insertion. Once a user believes applicator 10 is properly positioned, the user presses handle 26 toward body 14 to apply an actuating force to medical composition 12 via piston 24 and rod 60. The actuating force is transmitted from medical composition 12 to the interior of tip 16, tearing/opening portion 28 to place tip 16 in its open condition and allow medical composition 12 to be deployed from compartment 20 through tip 16 to the wound. In an embodiment, the user continues to apply an actuating force to handle 26 until piston 24, rod 60, and handle 26 reach the deployed position shown in FIG. 8.

Thus, in an embodiment, there is provided a method of treating a wound, comprising providing an applicator device, the applicator device having a body having a storage chamber for holding a composition, the storage chamber having an opening, and a delivery mechanism for dispensing the composition from the storage chamber through the opening in response to actuation by a user, the delivery mechanism comprising a delivery mechanism that is moveable between a compact position for storage and transport and a cocked position for deployment of the composition from the applicator, locating the opening of the storage chamber in proximity of the wound; and actuating the applicator to dispense the composition from the applicator into the wound.

It should be understood that while specific forms and constructions of embodiments have been shown and described herein, further modifications that will be seen by those skilled in the art and that may be desirable depending upon the particular requirements for each form of the applicator are contemplated. For example and without limitation, while several of the components have been disclosed as one-piece injection molded components, it may be desirable in some cases to make one or more of the components using other methods and/or to make one or more of the components a multi-piece assembly. By way of further example and without limitation, several of the components have been disclosed as having cylindrical shapes and/or circular cross-sections, but in some cases it may be desirable to make one or more of those components with something other than a cylindrical shape and/or circular cross-section. Accordingly, no limitation to a particular form, construction, shape, or material should be inferred to the invention unless expressly recited in an appended claim.

It is believed that applicator 10, as disclosed herein, is of particular use for combat medics and/or other emergency personnel for the local management of non-compressible hemorrhage, and is designed to function safely and reliably in a range of combat use scenarios. Applicator 10 is easy to operate, portable, and durable. It can provide a compact and lightweight construction that is impact and crush resistant and can function across extreme use environments and temperature. Applicator 10 provides the storage, transport, and rapid field deployment of medical composition 12, including the preferred hemostatic composition comprising a plurality of liquid expandable articles 13. Telescoping delivery mechanism 18 allows for rapid manual operation via a simple two-step process that requires minimal preparation by the user. Preferably, applicator 10 has a working volume of 90 cubic centimeters and can deliver an effective treatment dosage of 100 liquid expandable articles 13, which, in a suitable form, can expand up to ten times the initial volume in a few seconds. Tip 16 allows for safe and easy wound-track insertion, limited fluid ingress, and controlled delivery of medical composition 12. Applicator 10 requires minimal training and will fit standard training protocols for military medics. Delivery mechanism 18 provides both audible and tactile feedback when moved to the cocked position by virtue of the engagement of collet 66 with rod 60.

What is claimed is:

1. An applicator device, comprising:
a body having a storage chamber for holding a composition, the storage chamber having an opening; and
a delivery mechanism for dispensing the composition from the storage chamber through the opening in response to actuation by a user, the delivery mechanism comprising a delivery mechanism that is moveable between a compact position for storage and transport and a cocked position for deployment of the composition from the applicator.

2. The applicator device of claim 1, further comprising a tip at least partially covering the opening and maintaining the composition within the storage chamber.

3. The applicator device of claim 1, wherein the tip has a closed state in which the composition is maintained within the storage chamber and an open state in which the composition can be dispensed through the opening and the tip.

4. The applicator device of claim 1, further comprising the composition.

5. The applicator device of claim 4, wherein, in both the compact position and the cocked position, the composition is maintained in the storage chamber.

6. The applicator device of claim 1, wherein the delivery mechanism is a telescoping delivery mechanism.

7. The applicator device of claim 6, wherein the telescoping delivery mechanism includes a piston located within the storage chamber, the piston configured to move the composition in the storage chamber through the opening as the piston moves toward the opening from a stored position away from the opening.

8. The applicator device of claim 7, further comprising a user graspable handle for actuating the applicator.

9. The applicator device of claim 8, wherein the handle has a compact position adjacent the body with the piston located in a stored position, and has a cocked position in which the handle is spaced from the body and in operable engagement with the piston in the stored position.

10. The applicator device of claim 9, further comprising a rod connected to the handle for translating movement therewith, the rod having a first state in which the rod is free to translate relative to the piston and a second state in which the rod is connected to the piston to transmit an actuating force thereto from a user depressing the handle.

11. The applicator device of claim 10, further comprising a collet coupled to the piston, the collet having a plurality of fingers for engaging and coupling to a channel on the rod.

12. An applicator device, comprising:
a body having a storage chamber for holding a composition, the storage chamber having an opening;
a tip covering the opening of the storage chamber, wherein the tip has a closed state in which the composition is maintained within the storage chamber and an open state in which the composition can be dispensed through the opening and the tip; and
a delivery mechanism for dispensing the composition from the storage chamber through the opening in response to actuation by a user.

13. The applicator device of claim 12, wherein the tip has a convex shaped portion extending outwardly from the body and the opening.

14. The applicator device of claim 12, wherein the tip has a semi-spherical profile defined by a plurality of radially extending stiffening ribs.

15. The applicator device of claim 14, wherein one or more of the stiffening ribs are deformable to permit an opening within the tip to be formed or accessed to dispense the composition.

16. The applicator device of claim 14, wherein one or more of the stiffening ribs comprise a frangible portion to permit an opening within the tip to be formed or accessed to dispense the composition.

17. The applicator device of claim 16, wherein the frangible portion comprises a thinned section extending along the corresponding rib.

18. The applicator device of claim 12, wherein the tip comprises a small opening to serve as a propagation point for formation of a larger opening to permit dispensing the composition through the larger opening.

19. A method of treating a wound, comprising:
providing an applicator device, the applicator device having:
a body having a storage chamber for holding a composition, the storage chamber having an opening, and
a delivery mechanism for dispensing the composition from the storage chamber through the opening in response to actuation by a user, the delivery mechanism comprising a delivery mechanism that is moveable between a compact position for storage and transport and a cocked position for deployment of the composition from the applicator,
locating the opening of the storage chamber in proximity of the wound; and
actuating the applicator to dispense the composition from the applicator into the wound.

20. The method of claim 19, wherein the composition comprises a hemostatic composition.

21. The method of claim 19, wherein the composition comprises a plurality of liquid expandable articles.
